(19) United States
(12) Patent Application Publication
(10) Pub. No.: US 2025/0349411 A1
(43) Pub. Date: Nov. 13, 2025

(54) SYSTEMS AND METHODS FOR USE OF COMPUTER VISION AND ARTIFICIAL INTELLIGENCE FOR REMOTE PHYSICAL THERAPY

(71) Applicant: Electronic Caregiver, Inc., Las Cruces, NM (US)

(72) Inventors: David W. Keeley, Frisco, TX (US); Anthony Dohrmann, El Paso, TX (US); Robert Wood, Las Cruces, NM (US)

(21) Appl. No.: 19/275,829

(22) Filed: Jul. 21, 2025

Related U.S. Application Data
(63) Continuation-in-part of application No. 18/737,757, filed on Jun. 7, 2024, now Pat. No. 12,394,509.
Application No. 18/737,757 is a continuation of application No. 17/526,839, filed on Nov. 15, 2021, now Pat. No. 12,009,083.
(60) Provisional application No. 63/114,045, filed on Nov. 16, 2020.

(51) Int. Cl. G16H 20/30; A61B 5/00; G06F 3/048; G06N 20/00; G16H 10/60; G16H 30/00; G16H 40/67

(52) U.S. Cl. CPC G16H 20/30; A61B 5/744; G06F 3/048; G06N 20/00; G16H 10/60; G16H 30/00; G16H 40/67

(57) ABSTRACT

Systems and methods for physical therapy are presented herein. The technology provides systems and methods of utilizing computer vision, consumer computer and smartphone cameras, human pose estimation algorithms, and artificial intelligence to provide remote physical therapy. These technologies may comprise notifying an individual of a directed activity via an on-location at least one client device or console; identifying the individual with one or more sensors connected to or part of the at least one client device or console; utilizing the camera in the compute device to capture video of the directed activity; capturing the user's body location, position, orientation and movement through human pose estimation algorithms; utilizing artificial intelligence to personalize the performance of the directed activity; providing feedback to the user on the performance of the activity; and the capability to transmit results of the activity and analysis to a physical therapist or other wellness professional for further assessment and support.

### CROSS-REFERENCE TO RELATED APPLICATIONS

The present continuation-in-part application claims the priority benefit of U.S. Non-Provisional patent application Ser. No. 18/737,757, filed on Jun. 7, 2024, titled “Artificially Intelligent Remote Physical Therapy and Assessment of Patients,” which claims the priority benefit of U.S. Non-Provisional patent application Ser. No. 17/526,839, filed on Nov. 15, 2021, titled “Remote Physical Therapy and Assessment of Patients,” which claims the priority benefit of U.S. Provisional Patent Application No. 63/114,045, filed on Nov. 16, 2020, and titled “Methods and Systems for Remote Physical Therapy and Assessment of Patients”, all of which are hereby incorporated by reference in their entireties.

The present application is related to U.S. Pat. No. 10,813,572, issued on Oct. 27, 2020, and titled “Intelligent System for Multi-Function Electronic Caregiving to Facilitate Advanced Health Monitoring, Fall and Injury Prediction, Health Maintenance and Support, and Emergency Response”, which is hereby incorporated by reference in its entirety.

### FIELD OF INVENTION

The present technology pertains to remote physical therapy. In particular, but not by way of limitation, the present technology provides systems and methods of utilizing computer vision, human pose estimation algorithms, and artificial intelligence to provide remote physical therapy.

### SUMMARY

In various embodiments, the present disclosure is directed to methods carried out on a system and executed on one or more computing devices, which can be configured to perform particular operations or actions by virtue of having software, firmware, algorithms, hardware, or a combination thereof installed on the system and/or computing devices that in operation causes or cause the system to perform actions and/or method steps to enable remote physical therapy.

In some embodiments the present technology is directed to a system for remote physical therapy and assessment of patients, the system comprising: a) an at least one on-location sensor to capture and transmit visual data; b) an at least one on-location client device to display personalized instructions; c) an interactive graphical user interface to display the personalized instructions on the at least one on-location client device; d) a server system that includes: a user-interface, compute engine, storage, memory, and data infrastructure to analyze the visual data and produce updated personalized instructions; an at least one processor; a memory communicatively coupled to the at least one processor, the memory storing instructions executable by the processor; and e) a network; whereby the network is connected to the server system, the at least one on-location sensor and the at least one on-location client device. The on-location sensor may be a depth of field camera, standard red, green, and blue (RGB) camera, or RGB-D (depth) camera. The remote physical therapy system may include, connect to, and/or integrate with individual care management or electronic medical record system(s). The compute engine may also use data from various public and private data sources to produce updated personalized instructions, routines, movements, or physical therapy plans, separately or in addition to data the system collects itself. The network in this system may also be a content delivery network. The network may also connect to additional or fewer devices and systems. As described herein, there are many devices that can determine body location and movement.

Embodiments of the present technology may also be directed to systems and methods for physical therapy training and delivery (referred to herein as “PTTD”). PTTD incorporates assessment, training, delivery, recovery, and ongoing support. PTTD is a system comprising a directed care delivery platform, a remote rehabilitation management system designed for use in private pay, employee supported, and clinical reimbursement workflows.

Clinical reimbursement workflows include, but are not limited to, services such as RPM, RTM, CCM, musculoskeletal disease support, and pain management which refer to specific Medicare billing codes for services:

Recently introduced HCPCS codes, G3002 and G3003, specifically address Chronic Pain Management (CPM) services. Remote physical therapy, also known as telehealth physical therapy, is billed using a combination of specific CPT codes depending on the type of service provided. Codes like 97110 (therapeutic exercise), 97112 (neuromuscular re-education), 97116 (gait training), 97161-97164 (evaluations), 97530 (therapeutic activities), 97535 (self-care/home management training), 97542 (wheelchair management), 97750 (physical performance tests), 97755 (assistive technology assessment), 97760 (orthotic management and training), and 97761 (prosthetic training) can be used, often with the 95 modifier to indicate telehealth. Additionally, codes like 98975, 98977, 98980, and 98981 may be used for remote therapeutic monitoring (RTM) services.

RPM—Remote Patient Monitoring:

RPM involves using digital technologies to collect and transmit patient health data from outside traditional healthcare settings. Aspects include:

Devices: Blood pressure monitors, mobile phones, glucose meters, weight scales, pulse oximeters, etc.

Data collection: Physiologic data automatically transmitted to healthcare providers. Billing requirements: Minimum number of days of data collection per month, specific time thresholds for provider review, reimbursement codes including Current Procedural Terminology “CPT” codes and objective physiologic measurements.

RTM—Remote Therapeutic Monitoring:

RTM is newer and broader than RPM, covering non-physiologic data: Scope: Medication adherence, therapy compliance, cognitive behavioral therapy, musculoskeletal therapy data, both physiologic and non-physiologic patient-generated health data, treatment adherence and therapeutic response monitoring. For remote therapeutic monitoring (RTM) in physical therapy, several CPT codes are relevant. These include codes for initial setup and patient education (98975), device supply for musculoskeletal monitoring (98977), and treatment management (98980, 98981).

CCM—Chronic Care Management:

CCM provides comprehensive care coordination for patients with multiple chronic conditions, including care planning, medication management and coordination between providers. The focus is on care coordination and management of chronic conditions. These programs allow healthcare providers to deliver and bill for remote care services, expanding access while maintaining reimbursement for time and technology investments. Each has specific documentation requirements, patient eligibility criteria, and billing limitations that providers must follow for proper reimbursement.

The embodiment includes an artificial intelligence virtual physical therapy application that remotely delivers directed fitness regimens through a graphical user interface (GUI) with facilitated support via support from a professional such as a physical therapist, or a virtual agent. One embodiment of the virtual agent is an AI-driven avatar interface capable of delivering multimodal (i.e., visual, audio and verbal) physical therapy instructions, monitoring user motion and sentiment, and dynamically adjusting routines in response to real-time sensor feedback. Another embodiment could be an AI-driven chatbot or conversational agent while a third could be a human professional such as, but not limited to, a physical therapist, occupational therapist, exercise physiologist, care coach, or health and wellness coach.

The system integrates artificial intelligence into its human pose estimation and motion analysis software to provide remote physical therapy and health coaching for individuals seeking musculoskeletal (MSK) care, pain management, balance training, falls risk assessment and management, and other exercise support. The application may be used to supplement home fitness programs for preventive and rehabilitative physical therapy or by the user's physical trainers for sports cross-training. The application may allow a human professional to remotely deliver care plan to individuals and provides further insight into individual compliance, performance, and progress. This is accomplished by integrating machine learning algorithms (MLA) into human pose estimation and joint tracking motion analysis for user-compliance detection and kinesthetic progress. The application may include clinically validated physical therapy routines, exercise animations, and third-party access to joint-tracking data for external validation of users' regimens.

### BRIEF DESCRIPTION OF THE DRAWINGS

In the description, for purposes of explanation and not limitation, specific details are set forth, such as particular embodiments, procedures, techniques, etc. to provide a thorough understanding of the present technology. However, it will be apparent to one skilled in the art that the present technology may be practiced in other embodiments that depart from these specific details.

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 presents a schematic diagram of an exemplary system for remote physical therapy.

FIG. 2 presents a schematic diagram of an exemplary computing architecture that can be used to practice aspects of the present technology.

FIG. 3 presents the basic flow of one embodiment of a user profile set.

FIG. 4 presents one embodiment the initial interface to run motion analysis assessment in either clinical or validation modes.

FIG. 5. presents one embodiment of the motion analysis joint-tracking interface.

FIG. 6. presents an embodiment of an interface that allows clinicians to review multiple users' results.

FIGS. 7A-7D illustrate one embodiment of an interface for multi-user ground truth comparison.

FIG. 8 presents a flow diagram of a method to process video, image, and scene data as part of the remote physical therapy PTTD system.

FIGS. 9A and 9B present a diagram for a method for physical therapy and training delivered by an exemplary compute, data, and AI engine.

FIG. 10A presents one possible embodiment of a functional reach testing protocol carried out by the PTTD system for patients.

FIG. 10B presents one possible embodiment of the calculations carried out by the PTTD system and/or the AI engine for a functional reach test.

FIG. 11A presents one embodiment of an exemplary wall walking rehabilitation protocol.

FIG. 11B presents one embodiment of the calculations carried out by the PTTD system and/or the AI engine for a wall walking rehabilitation protocol for an individual receiving musculoskeletal (MSK) care.

FIG. 12 illustrates a computer system according to exemplary embodiments of the present technology.

FIG. 13 shows an exemplary method for remote physical therapy and guidance.

### DETAILED DESCRIPTION

Physical therapy is provided as a primary care treatment or in conjunction with other forms of medical and wellness services. It is directed to addressing illnesses, injuries, and trauma that affect an individual's ability to move or perform functional tasks. It can be an important component of many treatment regimens and is utilized in the treatment and long-term management of chronic conditions, illnesses and even the effects of ageing. It is also used in the treatment and rehabilitation of injuries, short-term pain, long-term pain, musculoskeletal (MSK) impairments, falls prevention and physical trauma. Some exemplary embodiments may also include personalized therapy progression plans generated by AI, sentiment-based engagement tracking to adapt to session style or tone, and/or patient or family interface options for reinforcement and adherence nudging.

Physical therapy may be composed of a number of components including the monitoring and/or assessment of patients, prescribing and/or carrying out physical routines or movements, instructing patients to perform specific actions, movements or activities, and scheduling short or long-term physical routines for patients; all these components designed to rehabilitate and treat pain, injury or the ability to move and perform functional tasks. Physical therapy may also contain an educational component directed to the patient and the patient's care circle.

Embodiments of the present technology provide systems and methods that enable physical therapy in some or all of its different forms to be undertaken and carried out remotely, from different locations and without any physical therapist or other individual being present with the patient.

While the present technology is susceptible of embodiment in many different forms, there is shown in the drawings and will herein be described in detail several specific embodiments with the understanding that the present disclosure is to be considered as an exemplification of the principles of the present technology and is not intended to limit the technology to the embodiments illustrated.

By making physical therapy remotely available to patients, the present technology enables a wide range of applications capable of addressing unmet needs in the areas of medical diagnostics, patient education, treatment, rehabilitation, communication and information sharing, and the like. There is generally a common need to find more cost-effective and scalable methods of assessing, monitoring and treating patients based on their medical history, future and long-term prospects, and their current physical status and abilities, as well as a need to deliver physical therapy in an accessible and standardized format to all patients, and in a variety of locations, with or without physical therapists or other individuals being physically present with the patient.

The term ‘patient’ is used to describe any individual that is using or intending to use or is prescribed the use of any embodiment of the systems and methods described herein, it may be used synonymously with the term ‘user’ or “individual”. Patient includes individuals receiving clinical care as well as non-clinical care. The term ‘care circle’ is used to describe individuals, organizations or entities that may be assigned either by the patient or by other means to be notified and informed of the patient's status, progress or need for immediate attention and/or help.

The remote physical therapy system provides and can incorporate and utilize different forms of motion detection, monitoring and tracking capabilities, in conjunction with analysis that may be executed and provided by artificial intelligence (AI) to both enhance the capture of audio-visual and other motion data as well as to provide analysis of the captured motion detection data and other available data. The AI engine may utilize amongst other factors, performance metrics from a patient carrying out physical therapy routines, or patient movement measurements, as variables to determine or calculate performance factors and metrics, patient health status, or other indicators of the patient's physical or mental state or wellbeing (all these collectively referred to as “patient state”). This system may also be able to integrate with, and read and/or write data to, electronic medical record systems of patients, and incorporate or utilize these records in analysis and other computations. A patient's historical data, both captured by the system and from external records, may serve as a baseline from which the system and AI engine uses to determine past, current, and future performance metrics or to provide insights into the health status of a patient. The assessment and analysis may also be carried out by analyzing metrics of performance, recovery, and fitness. The use of an AI engine is not strictly necessary.

Some embodiments of the system deliver routines to the patient's client device and display it with a graphical user interface, which may be that of an interactive avatar. The client device may feature a virtual physical therapist, wellness coach, or caregiver powered Artificial Intelligence based gait and physical therapy console, or a display device such as a cellular phone, tablet, computing device, and/or augmented reality or virtual reality headset/device or other audiovisual technology device. The interactive avatar may be able to perform routines or movements and provide instructions, feedback, information, communication, engagement or perform examples of physical therapy movements or routines for the patient or their care circle. The presence or use of the virtual avatar is not required.

Some embodiments of the system may also be incorporated into smart home technologies, homecare, or other software applications or electronic devices that may assist in monitoring, observing, and ensuring compliance, or otherwise assist in detecting movement, measuring performance of patients, or even displaying the graphical user interface and/or interactive avatar. One or more plug-and-play input and output devices may also be used and be incorporated into the system.

Motion capture, depth capture, body position capture, movement capture, skeletal tracking, detection of sounds, or the capture of infrared data may be undertaken by components embedded within a dedicated console or by plug-and-play or other devices that can be added to the system as desired, these devices may include microphones, cameras, including depth of field, infrared or thermal cameras, or other motion or image capture devices and technology.

Additional devices may include, however not limited to:
Depth Cameras;
Structured Light Cameras (e.g., Microsoft Kinect, Intel RealSense series);
Time-of-Flight (ToF) Cameras (e.g, Azure Kinect DK, PMD CamCube);
Stereo Vision Cameras (e.g., Intel RealSense D435, ZED cameras by Stereolabs);
LiDAR Cameras (solid-state LiDAR systems, spinning LiDAR units);
Specialized Motion Capture Cameras:
Infrared Motion Capture Cameras (e.g., OptiTrack Prime series, Vicon Vantage cameras);
High-Speed Cameras for detailed movement analysis (e.g., Phantom TMX series, Photron NOVA);
Marker-Based Tracking Cameras (designed to track reflective markers);
Computer Vision Cameras:
RGB (red, green, blue) Cameras
RGB-D (red, green, blue plus depth) Cameras
RGB Cameras with AI Processing (standard cameras with pose estimation software);
Multi-Spectral Cameras (combining visible and infrared spectrums);
360-Degree Cameras (for full environmental capture);
Consumer/Prosumer Options;
Smartphone Cameras (with depth sensors like iPhone's TrueDepth, Android ToF sensors);
Webcams with Depth (e.g., Intel RealSense webcams);
Gaming Cameras (PlayStation Camera, Xbox Kinect);
Action Cameras (GoPro with stabilization and motion tracking);
Professional Systems:
Volumetric Capture Cameras (arrays of synchronized cameras);
Facial Motion Capture Cameras (specialized for facial tracking); and/or
Inertial Measurement Unit (IMU) Camera Systems (combining visual and inertial data).

Various combinations of the above devices and/or sensors may also be employed. For example, multiple camera types (e.g., Red, Green, Blue “RGB”+depth, stereo+Inertial Measurement Unit “IMU”) may achieve more accurate and robust tracking across different lighting conditions and environments. Some embodiments may incorporate air handling, air correction, skeletal tracking software and other detection and monitoring technologies.

Additionally, gyroscopic sensing from mobile devices, head mounted smart glasses with gyro sensors and hearing aids may also be employed.

Embodiments of the system may be able to detect movement, general health status and indicators, adverse reactions to drugs, sudden or rapid movements including but not limited to seizures, falls or heart attacks, or other changes in the physical or mental state of the patient based on visual, motion detection, skeletal tracking, sound, or other forms of captured data. It may detect or determine the individual's state based on long or short-term analysis and/or calculations in conjunction with motion detection and/or analysis of a patient. It may also detect and/or determine the patient's state from data not directly collected or obtained from the physical therapy monitoring and assessment system. For example, data from the user's records, medical history and of drug use may all be used.

In some embodiments, the system may be able to detect specific illnesses, diseases, deformities, or ailments suffered by the patient. One example could be detecting a parkinsonian shuffle from the gait velocity, time-in swing, time in double support, cadence, inverted pendulum measurement, or festination of a patient.

In various embodiments a notification or form of communication is sent to the patient's care circle, to notify them of changes in the patient state, non-compliance with scheduled routines or when certain movement(s) are detected. The form of notification or communication may be set or may be designated by the system depending on the severity of the detected motion or status of the patient. Notification may be carried out using any form of communication including but not limited to digital, electronic, cellular, or even voice or visual, and may be delivered through a monitor, television, electronic device, or any other interface that is able to communicate with or notify the patient or their care circle.

In various embodiments, the system may be deployed in hospitals, medical offices and other types of clinics or nursing homes, enabling on-site and live motion detection and analysis of patients. One example may be the detection of a patient that walks into a hospital, where the characteristics and datapoints collected from the patient's motion inside the premises are captured and analyzed, and a report is produced and transmitted/communicated to the designated physician. The physician seeing the patient will have up-to-date information prior to the visit that will inform the physician to look for certain symptoms, or possible health issues that were detected or red-flagged from the analysis of the patient's motion and/or other captured characteristics. This enables the physician to undertake tests or ask more detailed questions based on the indicators and report provided by the system.

In some embodiments, the system may prescribe specific movements or physical therapy routines, from a library containing a catalogue of physical therapy movements, routines, and regimens, to provide musculoskeletal care, treat or rehabilitate patients or reduce their pain from injuries, to reduce future health risks, falls risk and other physical problems or potential for injuries. This library may be stored in a database accessible by users and other stakeholders. The prescribed movements and/or routines may be personalized for each person based on both captured personal data as well as the patient's external medical records or data. Artificial intelligence may be utilized to prescribe or provision specific movements, routines, or regimens. Artificial intelligence or machine learning may be used to detect and assess the patient's or user's health status, general health indicators and patient state, and prescribe and alter movements, routines and/or physical therapy plans accordingly. Artificial Intelligence may also access databases or other external information repositories and to incorporate that data when tailoring, customizing, and provisioning movements, routines or plans according to the patient's goals or needs.

In some embodiments artificial intelligence uses the total information captured from all patients to devise new physical therapy movements, routines or plans it assesses to be beneficial to a specific patient, these movements or routines may be created and then added to a library containing a catalogue of physical therapy routines or movements. Devising new routines or movements allows for specific and more precise treatment plans to be delivered to each patient. These treatment plans may then be collected or organized into standardized plans that are delivered to other patients that possess certain common factors or indicators. Prescribed changes may then be sent to the patient's care circle.

A library containing all movements and routines/regimens may be updated by adding new routines or movements and may be accessed to update individual patient routines. Each movement, or routine comes with its own preset and calculated assessment metrics, performance variables, as well as associated notifications and instructions.

### Physical Therapy Training and Delivery

### Part 1. Individualized Provisioning

Various embodiments of the invention utilize an artificial intelligence virtual physical therapist, assigned routines, and a graphical user interface (GUI) with image overlays and real-time feedback to the user for remote physical therapy. Remote physical therapy is also referred to in this document as physical therapy training and delivery (PTTD). PTTD incorporates assessment, training, delivery, recovery, and ongoing support. PTTD delivers an exercise regimen or routine through a GUI. The exercise regimen can be individually tailored to improve a user's health status or condition by providing a customized approach to provisioning the virtual exercise programs. Provisioning of the user's regimen can be done by the individual or a qualified third party. A qualified third party can be, but is not restricted to, a user's physician, physical therapist, wellness coach, or any other responsible party or member of the care circle. Provisioning of the regimen or routine includes, but is not restricted to, the selection of exercise(s), the number of sets, the number of repetitions, and the regimen schedule. The user may choose their programmed exercises from a non-relational database that connects to the GUI. The objects in the database serve as inputs to a schema that is programmed by the individual or third party. The schema pulls appropriate animations of the virtual caregiver to the user interface based on provisioned user inputs. Once a regimen is selected or added to the user's goals, the regimen is stored to the user's profile. The primary objectives of PTTD are to improve patient mobility, reduce the need for ongoing musculoskeletal care, improve functional health, or address pain management. Remote physical therapy can also be used in a cross-training environment to improve athletic performance of individuals and/or athletes.

### Part 2. Clinical Development of Exercise Directory

### Part 2.1 Exercise Directory

Physical therapists (PT) collaborate with PTTD developers to continually build and improve upon an exercise directory and animation motion capture footage. A licensed PT may provide developers with accredited preventative and rehabilitative exercises for virtual instruction. The exercises are stored to a database for users' individualized provisioning. Each exercise in the database can be based on, but is not restricted to, anatomical or injury classification (e.g., hip strengthening or rib fracture rehabilitation.)

Part 2.2. Motion Capture with Licensed Physical Therapist

In various embodiments, the database couples each exercise with its appropriate virtual caregiver animation. This animation serves as the set of exergame instructions for the user to follow. The avatar leads individuals through exercise. The motion capture (mocap) process for the animation is done utilizing mocap software. A developer in a mocap suit is recorded by cameras while performing exercises provided by the licensed PT. The PT supervises the developer through the motion capture process. The motion capture footage serves as the skeletal blueprint onto which the virtual caregiver's animation can be rendered. This ensures that the virtual caregiver instructs the patient with proper clinical form and modifications.

### Part 3. Joint Tracking and Motion Analysis Integration

### Part 3.1 Joint Tracking and Motion Analysis

PTTD records and measures a user's compliance with their provisioned regimen or routine. This is accomplished by joint tracking and motion analysis software that is integrated with a depth camera. The user's movements are recorded via a non-invasive depth tracking software that collects real-time spatial location of joints across a three-dimensional axis. Changes in the spatial location are calculated into a multitude of variables such as, but not limited to, the number of repetitions, cadence, gait velocity, stride length, range of motion, and time to completion. Prior to beginning the regimen, the user will go through a calibration routine that collects the user's anatomical ground-truth data. This allows the motion analysis software to track the user's movements with greater precision and accuracy. The user's ground-truth data is processed through the motion analysis software and labeled appropriately for future analysis. The user is prompted according to the provisioned regime to begin the prescribed activity. The user begins the activity, and their movements are recorded simultaneously as the provisioned regimen streams. All footage of users is made non-identifiable through a gray scale filter.

Part 3.2 Joint-Tracking with Analytics and Streaming Access

Each activity stream that is recorded by the camera gets appended on to the user's profile in a non-relational database. Each time a new activity stream is stored to the user profile, a machine learning algorithm (MLA) is triggered for analysis on joint-tracking data. Embodiments include the use of human pose estimation algorithms as a component the MLA. The MLA compiles the user's calibrated ground truth data as a baseline to compare future joint-tracking data. The MLA can be, but is not limited, to, supervised or unsupervised models that interpret when the user's kinesthetics are anomalous to their usual patterns. Anomalies detected by the MLA are classified as improvements or declines that are visualized on a GUI. Another option for additional insight is access to the user's stored activity data without MLA analysis. Upon user authorization, stored activity data can be available for playback. This allows the user or an authorized third-party to review streaming footage to validate compliance and kinesthetic progress at their own discretion and provides another source of ground-truth for the models.

The motion capture and joint tracking can be accomplished utilizing a variety of camera types combined with human pose estimation algorithms and additional application of artificial intelligence to personalize the results for the individual. The use of a depth camera is an embodiment but is not required. Standard commercial RGB cameras, RGB-D cameras, smartphone cameras, and computer cameras with human pose estimation algorithms and artificial intelligence are among other embodiments.

### Part 4 Data Storage

### 4.1 Individual Database

Another valuable component of remote physical therapy is the cloud-based data store. Gait and posture are unique to an individual based on personal characteristics and features such as medical history, age, and gender. One impediment in machine-learning based motion analysis is obtaining ground-truth data. What could be interpreted as anomalous movement for one user could be classified as an improvement for another if compared to a general population average. To address this, remote physical therapy implements two methods of analysis: an individualized model, and a population-based model. Each time motion analysis is triggered for an individual, the data is stored to their user database. The individualized model has a user database that restricts its analysis to ground-truth data supplied only by the individual. The progress reports from the individual's database are compared to their personal ground-truth data set. As the user increases their remote physical therapy usage, the motion analysis and human pose estimation algorithms tailor insights to their individual baselines with the new datapoints, and this additional data allows the algorithm to gain additional insight into developments or progress in the user's kinesthetics. This allows the algorithm uses for remote physical therapy to continually retrain itself for improved accuracy.

### 4.2 Population Data Lake

From these individual databases, data is pulled into a general population data lake to provide further macro-scale or big data insights. Authorization to access the data lake can be granted to any individual or organization through an ordering process. This makes ground-truth data available to those who do not have access to the physical hardware and the infrastructure that goes into collection of motion analysis data but wish to perform research and development using the data lake's features. All data is encrypted at rest and in transit.

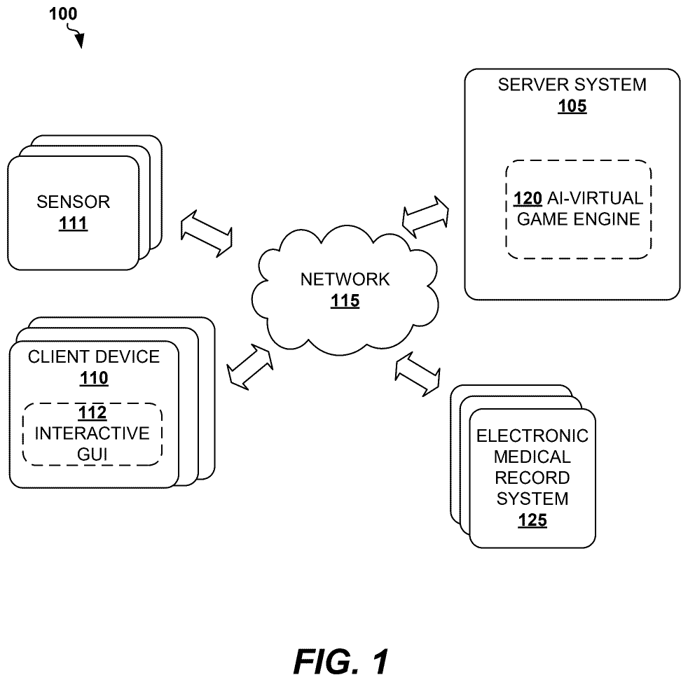

FIG. 1 illustrates an exemplary generalized architecture for practicing some embodiments of the remote physical therapy system.

The remote physical therapy system 100 includes a sensor 111 enabled to capture data, a client device 110 that displays an interactive avatar through a graphical user interface 112, a server system 105 that includes a compute, data analysis, and AI engine 120 which provides the functionality of the system and all of its embodiments as described throughout this document. The system may also include a data storage, analysis, and interface system 125. The different components of the system are connected via a network 115.

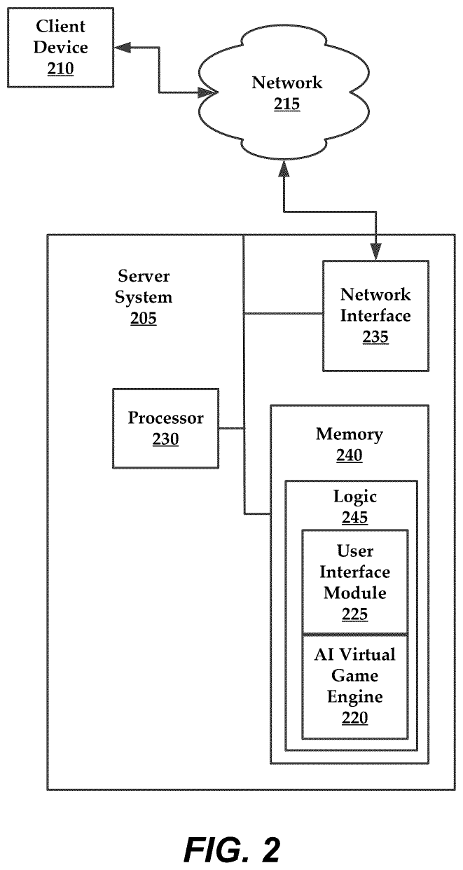

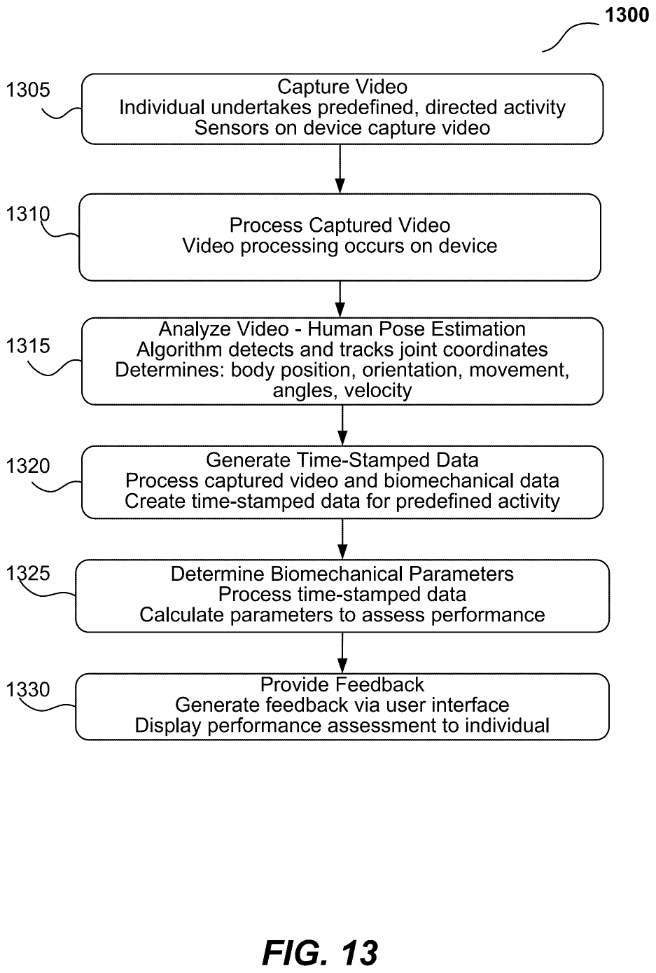

FIG. 2 illustrates an exemplary architecture for practicing aspects of the present technology that provides a more detailed view of aspects of the system. The architecture comprises a server system, hereinafter “system 205” that is configured to provide various functionalities, which are described in greater detail throughout this document. Generally, the system 205 is configured to communicate with client devices, such as client device 210. An example of a computing device that can be utilized in accordance with the present technology is described in greater detail with respect to FIG. 13.

The system 205 may communicatively couple with the client 210 via a public or private network, such as network 215. Suitable networks may include or interface with any one or more of, for instance, a local intranet, a PAN (Personal Area Network), a LAN (Local Area Network), a WAN (Wide Area Network), a MAN (Metropolitan Area Network), a virtual private network (VPN), a storage area network (SAN), a frame relay connection, an Advanced Intelligent Network (AIN) connection, a synchronous optical network (SONET) connection, a digital T1, T3, E1 or E3 line, Digital Data Service (DDS) connection, DSL (Digital Subscriber Line) connection, an Ethernet connection, an ISDN (Integrated Services Digital Network) line, a dial-up port such as a V.90, V.34 or V.34bis analog modem connection, a cable modem, an ATM (Asynchronous Transfer Mode) connection, or an FDDI (Fiber Distributed Data Interface) or CDDI (Copper Distributed Data Interface) connection. Furthermore, communications may also include links to any of a variety of wireless networks, including WAP (Wireless Application Protocol), GPRS (General Packet Radio Service), GSM (Global System for Mobile Communication), CDMA (Code Division Multiple Access) or TDMA (Time Division Multiple Access), cellular phone networks, GPS (Global Positioning System), CDPD (cellular digital packet data), RIM (Research in Motion, Limited) duplex paging network, Bluetooth radio, or an IEEE 802.11-based radio frequency network. The network 215 can further include or interface with any one or more of an RS-232 serial connection, an IEEE-1394 (Firewire) connection, a Fiber Channel connection, an IrDA (infrared) port, a SCSI (Small Computer Systems Interface) connection, a USB (Universal Serial Bus) connection or other wired or wireless, digital, or analog interface or connection, mesh or Digi® networking.

The system 205 generally comprises a processor, 230, a network interface 235, and a memory 240. According to some embodiments, the memory 240 comprises logic (e.g., instructions) 245 that can be executed by the processor 230 to perform various methods. For example, the logic may include a user interface module 225 as well as an AI engine 220 which includes data aggregation and correlation (hereinafter application 220) that is configured to provide the functionalities described in greater detail herein including systems and methods of remote physical therapy.

It will be understood that the functionalities described herein, which are attributed to the system 205 and application 220 may also be executed within the client 210. That is, the client 210 may be programmed to execute the functionalities described herein. In other instances, the system 205 and client 210 may cooperate to provide the functionalities described herein, such that the client 210 is provided with a client-side application that interacts with the system 205 such that the system 205 and client 210 operate in a client/server relationship. In some embodiments, complex computational features may be executed by the server 205, while simple operations that require fewer computational resources may be executed by the client 210, such as data gathering and data display.

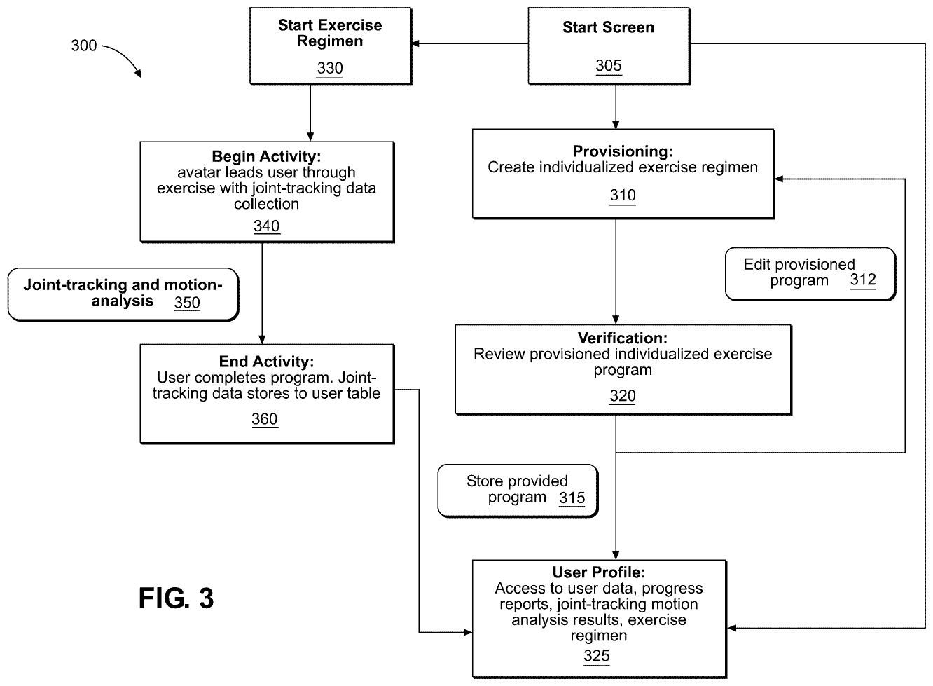

FIG. 3 is a diagrammatical representation that illustrates the basic flow of a user profile set 300 in the PTTD system. The user logs in to remote physical therapy application and is presented with a Start screen 305 with the option to either provision 310 or begin 330 an exercise regimen. Exercise regimens can be triggered manually by the user or through a reminder. All user activity data is stored to their user profile. Provisioning 310 will allow the user to create a new (or modify an existing set 312) individualized exercise or physical therapy rehabilitation set. Provisioned exercise regimens can also be tailored by an individual or third-party (such as the individual's health coach, physical therapist, or care circle). The individual must be verified first 320 by the system, through image, voice or other identification known in the art. Once the user's identity is verified or confirmed 320, the patient may access the User Profile 325 that contains information and data, including but not limited to historical progress reports, joint-tracking motion analysis results, exercise regimen(s), diet information, medical and health records, prescribed medications, as well as supplementation and other health related activities or logs that the individual has been keeping or that has been kept for the individual by a computing device, care circle, physician, therapist or smart IOT and/or tracking technologies. In various embodiments the Start screen 305 instantly takes a user to a User Profile screen 325. The provisioned program from step 310 may also be stored 315 into the user profiles. When a user starts or chooses to begin 330 an exercise regime, the activity begins 340, in one embodiment an avatar or conversational agent demonstrates to, or leads the user through exercise or rehabilitative movements and therapy. As the user undertakes the activity, the remote physical therapy system collects human pose estimates, joint-tracking, and motion-analysis information 350. In various embodiments when the data is collected it is also analyzed in this step, locally, via a network, on a server, or on a cloud server system. Once the user ends activity 360, whether completed or not, the data collected is stored to the user profile, user tables, databases, or all these.

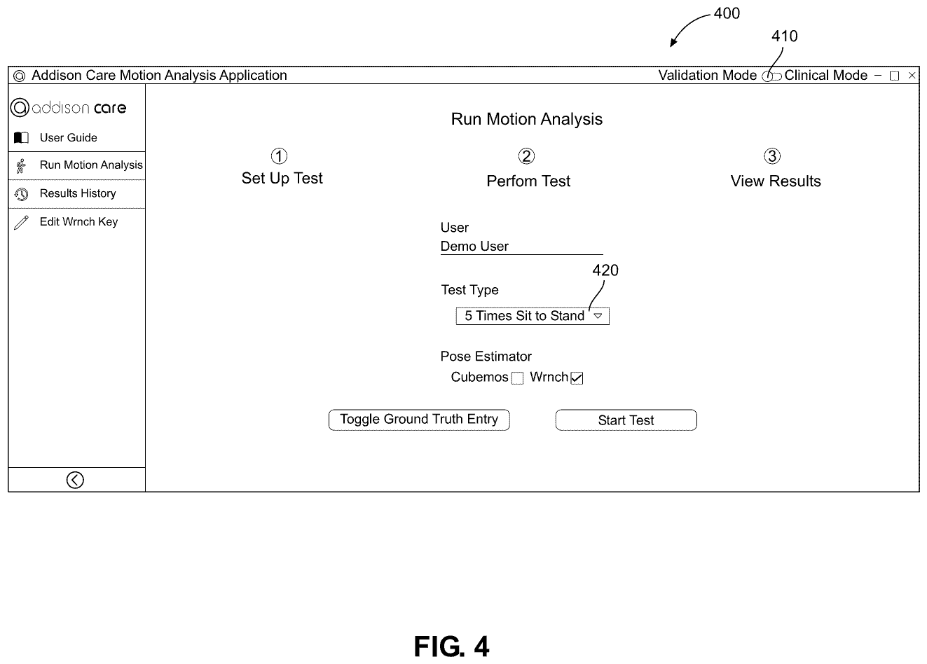

FIG. 4 illustrates the Run Motion Analysis Interface 400. This is the initial interface to run motion analysis assessment in either Clinical or Validation modes. Motion analysis assessment usually comprises 3 steps, setting up the test, performing the test and viewing results. Clinical mode is for performing the specified assessment to measure patient progress. Validation mode is available to clinicians who wish to review user data. In some embodiments a button or slider 410 may be toggled to switch between Validation and Clinical modes. The user may also have the option to select the type of test 420.

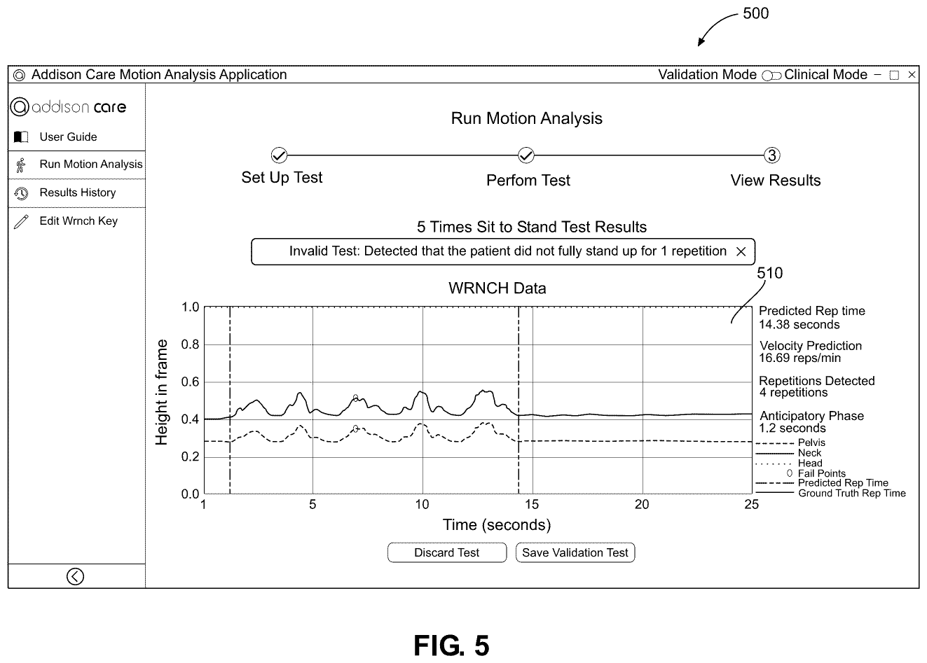

FIG. 5 illustrates the motion analysis joint-tracking interface 500. The joint-tracking assessment 510 measures the changes in the position of the pelvis, neck, and head, and these are plotted against time to measure number of repetitions, velocity, and time to completion.

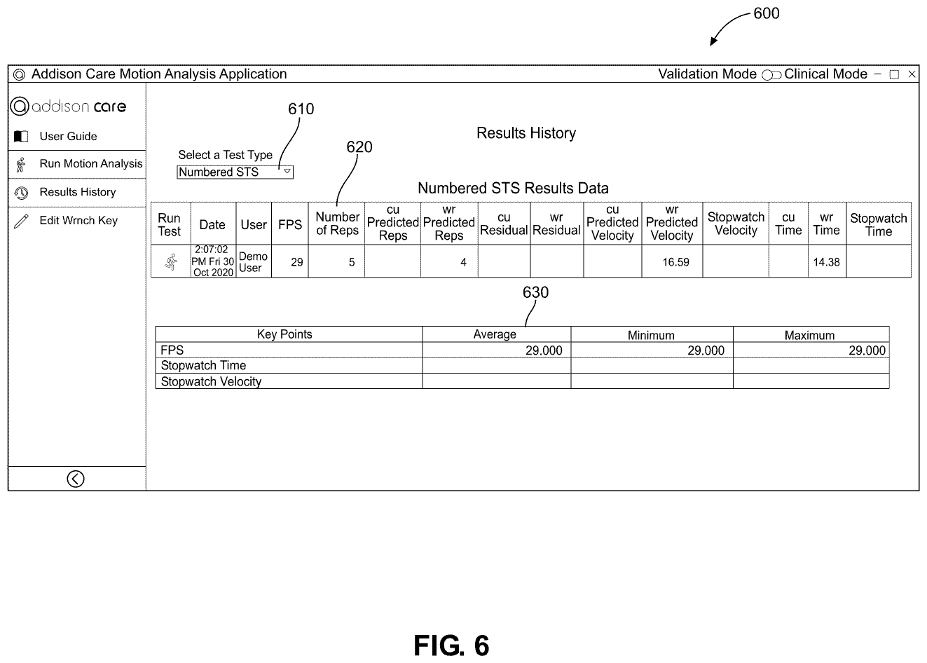

FIG. 6 illustrates a Validation Mode User Profile Activity Access interface 600. This is an interface that allows clinicians and other individuals involved in providing remote physical therapy to review multiple users'/patients' results. A user of the interface or clinician may select the test 610 that the data is sought for. The user's ground-truth data are processed through the motion analysis software and labeled appropriately for future analysis. The data may be presented in analysis tables 620 and 630.

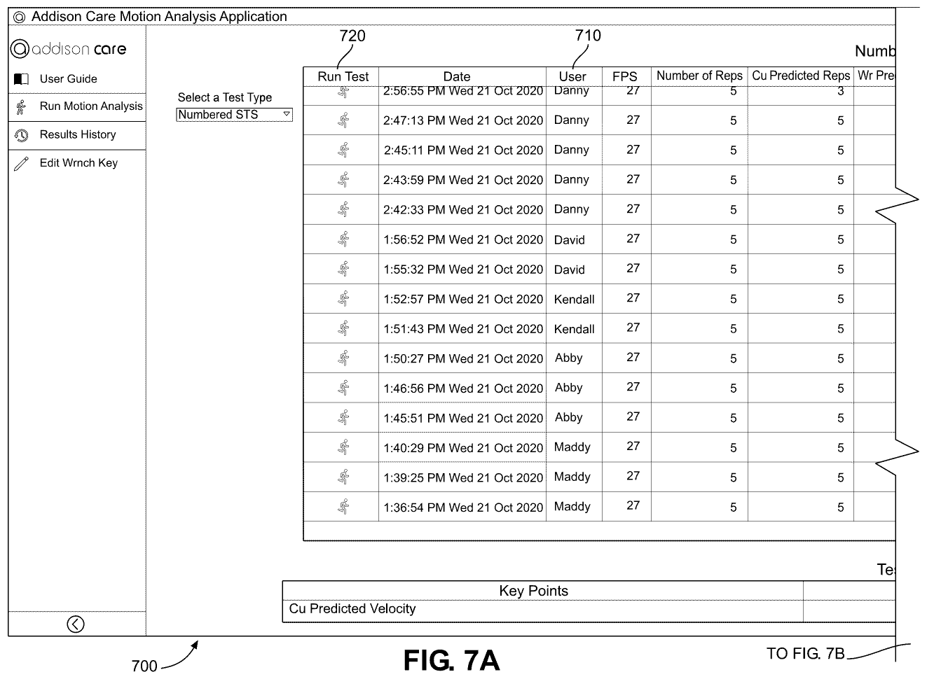

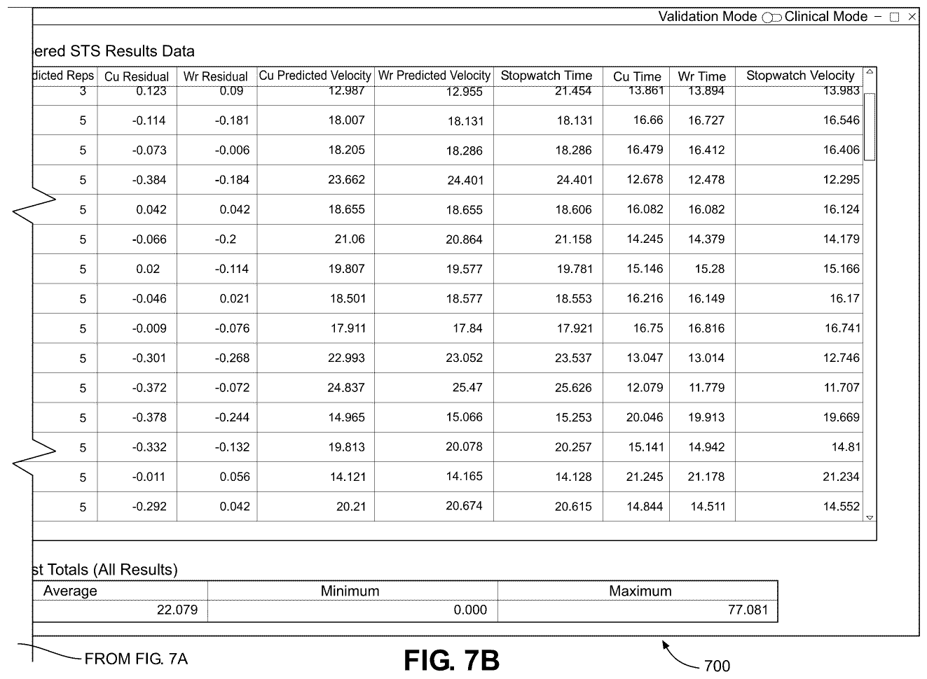

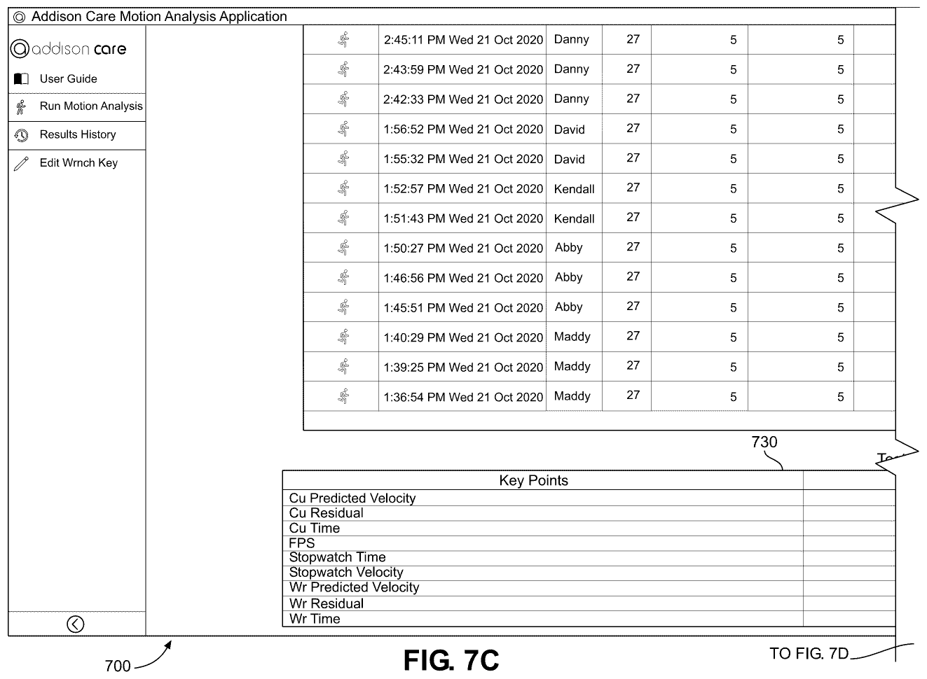

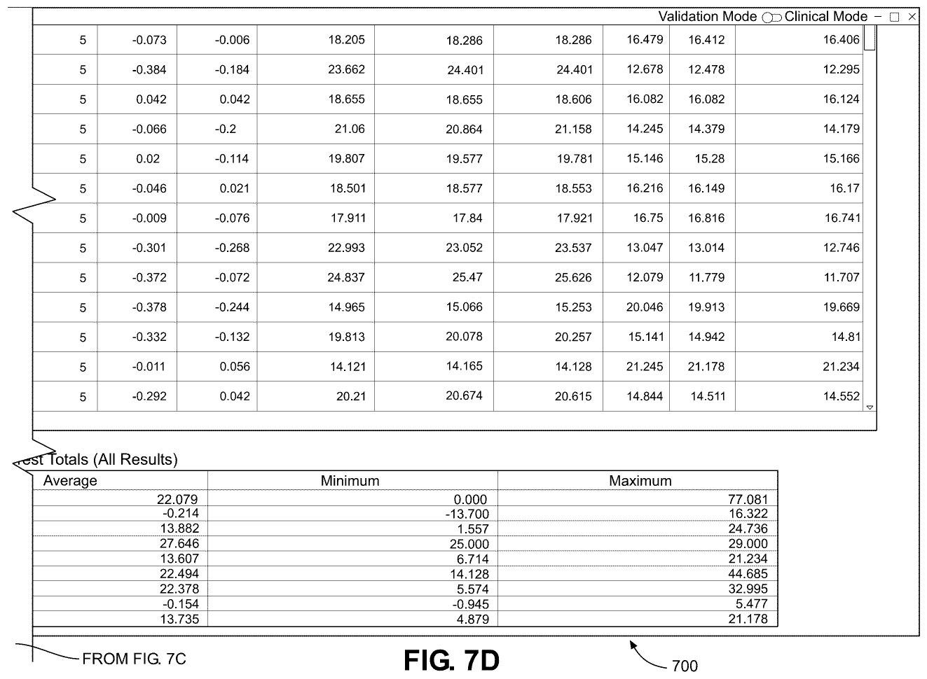

FIGS. 7A-7D illustrate the interface for multi-user ground truth comparison 700. This is a ground-truth comparison interface that allow clinicians and other to compare user results 710 of one or multiple users for further insight and analysis, and for each test 720 undertaken. In several embodiments, an MLA compiles the users' calibrated ground truth data as a baseline to compare future joint-tracking data. FIGS. 7A and 7B show the top half of the interface screen, while FIGS. 7C and 7D show the bottom half of the interface screen. A Test Totals table 730 showing all results collected, presents derived and analyzed data from all tests run.

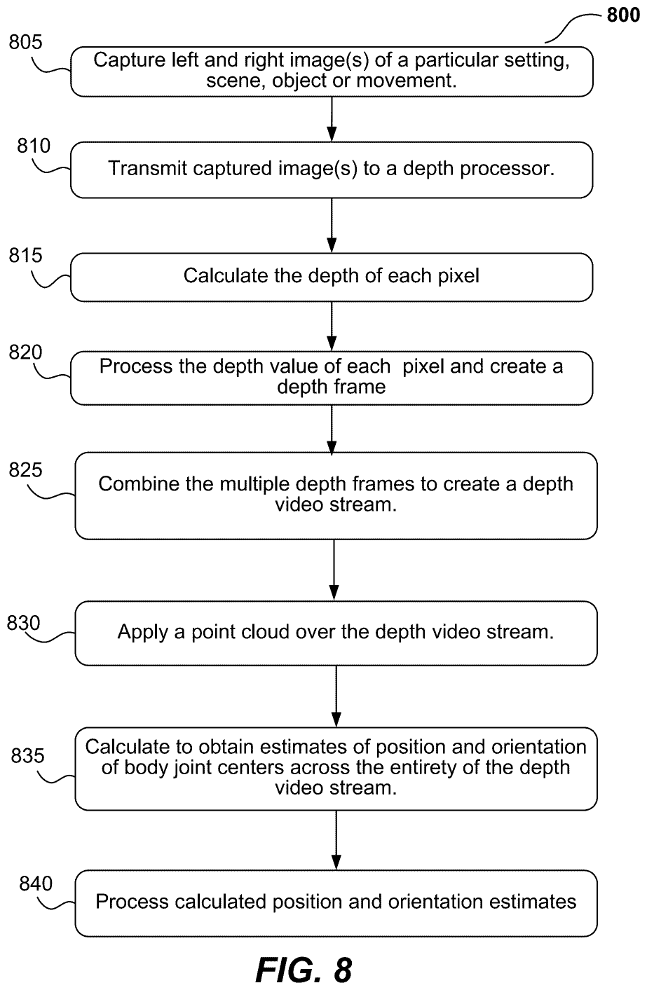

FIG. 8 presents flow diagram of a method 800 to capture and process body position, movement, and depth video and images and scene data as part of the remote physical therapy system. In several embodiments a motion capture module is deployed along with a depth camera. In some embodiments a human pose estimation algorithm is deployed along with a non-depth camera. In some embodiments the motion capture module may be contained in, be part of, or connected to a compute data, and AI engine. The camera may calculate depth in various ways including and not limited to stereo vision technology. Stereo vision technology is utilized by having the camera capture 805 left and right image(s) of a particular object or movement. The captured scenes and scene data are transmitted 810 (for example in pixels) to an internal or external image processor. The image processor then uses software, human pose estimation algorithms, and MLAs to calculates 815 the depth of each pixel. The depth values of each pixel are then processed by an imager to create a depth frame 820. With the combination of multiple depth frames create a depth video stream 825. Specially designed software then applies 830 a point cloud over the depth video stream, which allows for estimates of position and orientation of body joint centers to be calculated 835 across the entirety of the depth stream. Finally, position and orientation estimates are then processed 840 using accepted calculations known in the art.

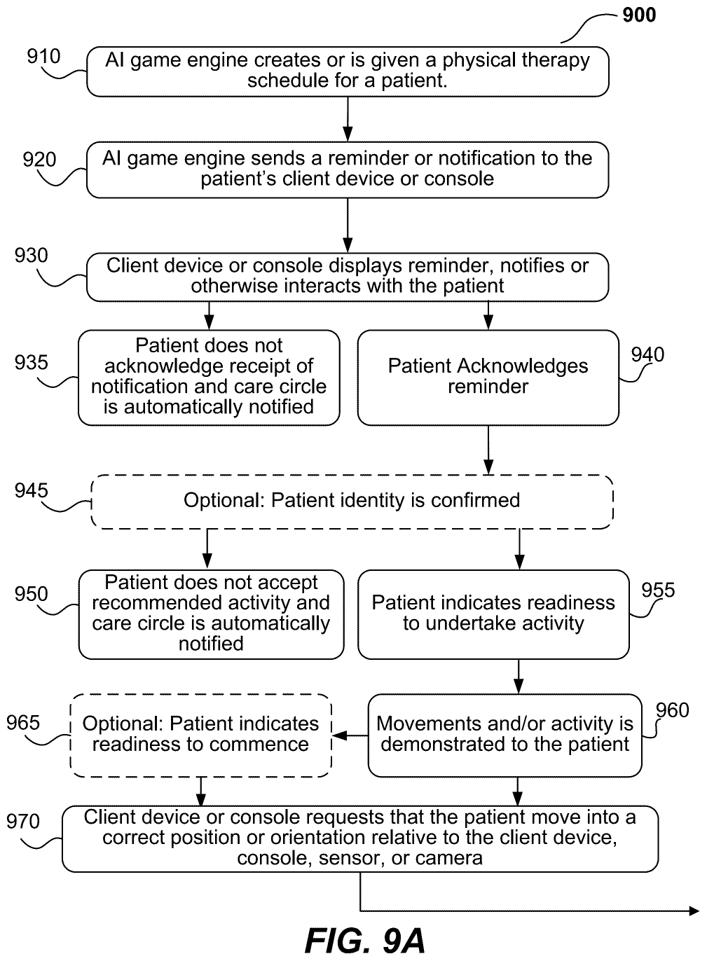

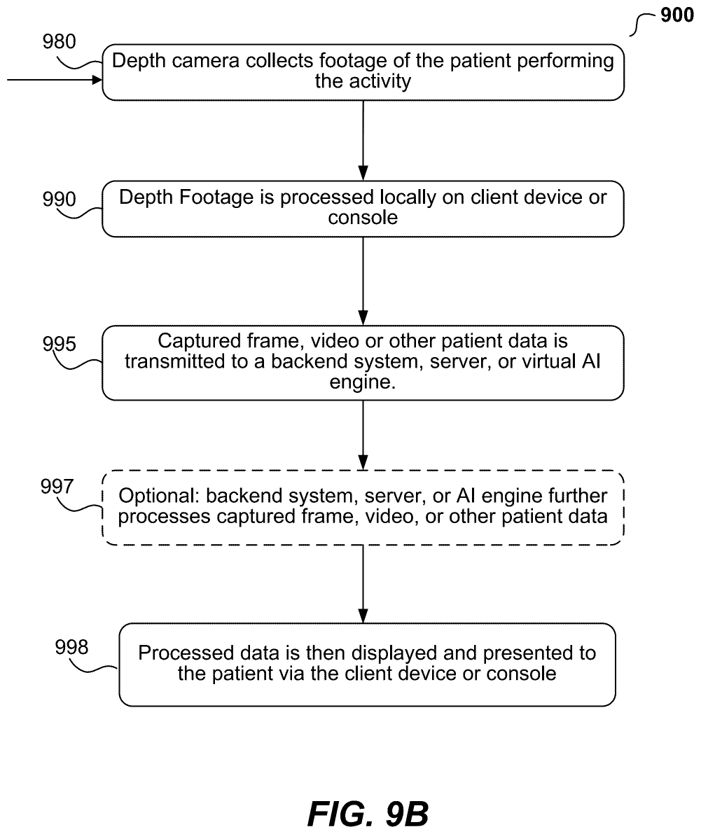

FIG. 9A presents a diagram for a method 900 for Physical therapy and training delivered by an exemplary compute, data, and AI engine. In some embodiments, the AI engine may be stored, run, or executed on a server. In other embodiments the AI engine may be stored, run, or executed on a serverless, or cloud-based systems, or alternatively on a client, console or computing device, or a combination of all of these. In several embodiment the AI engine (“AI”) executes the PTTD by scheduling one or more physical therapy activities for a user and provide reminders for exercises to be performed.

The AI game or compute engine creates or is given a physical therapy schedule for an individual or patient 910. The AI game engine sends a reminder to the patient or the patient's client device or console 920. The client device or console delivers the reminder or notification to the patient 930. The patient acknowledges the reminder 940. In some embodiments, the patient may have to confirm their identity 950 to the client device or console. Only after confirmation of the patient's identity does the client device or console receive and/or display the patient's routines or other private data. The console or client device may confirm the identity of the patient via facial recognition or via other means. If the patient chooses not to perform the physical therapy activity, an alert is generated, and the patient's care circle is notified 945. Otherwise, the patient chooses, accepts, or confirms that the activity will be performed to the client device or console 955. The movements are demonstrated 960 to the patient or user via the client device or console, in many embodiments with an avatar or conversational agent. The user then indicates that he/she is ready to begin demonstrated activity 965. The client device or console requests 970 that the patient move into a correct position or orientation relative to the client device, console, or camera.

FIG. 9B continues from FIG. 9A where a camera collects video footage, images, and frames of the individual performing the activity 980. In some embodiments, the video footage is processed 990 locally on the client device or console to identify quantitative markers of the activity. One way the camera may process 990 the image data is via the method presented in FIG. 8. Data is transmitted to the server or other backend system 995. Captured patient data which may be comprised of captured image, frame, video, performance, biometric or any other data may be further processed by the backend system, server, algorithms, or AI engine 997. Processed data is then displayed and presented 998 to the user via the client device or console. The data can be depicted in a variety of forms and formats to provide feedback to the user. This feedback may include analysis of trends, changes in activity performance, and an assessment score.

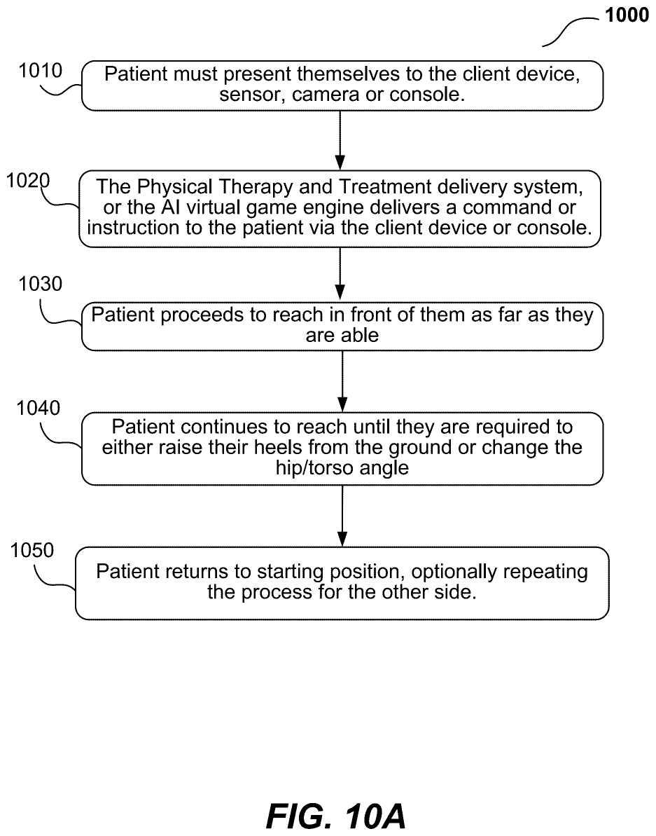

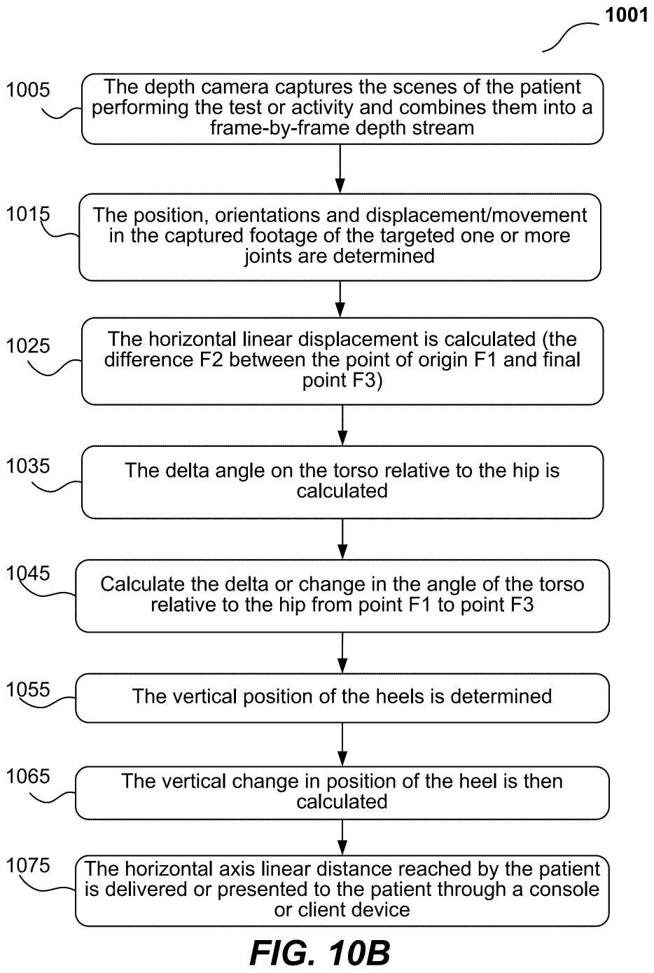

FIG. 10A presents one possible embodiment of a functional reach testing protocol 1000 carried out by the PTTD system for patients. In this embodiment, to perform a functional test, for example a reach test, the patient must present themselves 1010 in front of the console or client device and/or the camera, for example by standing in front of the camera, making sure that one or more of the following required joints are visible to the camera, right heel, left heel, right hip, left hip, right wrist, left wrist, torso, neck and head. Of course, the joints that must be visible to the camera depend on the requirements of each test. Each test may require one, some, or all the joints to be visible. The PTTD system delivers a command or instruction 1020 to the user, which may be a voice instruction, the patient raises either left or right arm and proceeds to reach in front of them as far as they can 1030. Patient continues to reach 1040 until they are required to either raise their heels from the ground or change the hip/torso angle. Patient then returns to starting position 1050, optionally repeating the process for the other side.

FIG. 10B presents one possible embodiment of the calculations 1001 carried out by the PTTD system and/or the AI engine for the functional reach test discussed in FIG. 10A. The camera captures the scenes of the patient performing the test or activity and combines them into a frame-by-frame data stream 1005. One way this may be undertaken is presented in more detail in FIG. 8. The positions, orientations, displacement, and movement of the required one or more joints are determined 1015 by the system. These joints may include any one or more of, and not limited to: the right heel, left heel, right hip, left hip, right wrist, left wrist, torso, neck and head. The horizontal linear displacement of the wrist joint from point F1 to point F3 is calculated 1025 by the difference F2 between the two points F1 and F3 (i.e., F3−F1=F2). The delta angle of the torso relative to the hip is calculated 1035. The delta or change 1045 in the angle of the torso relative to the hip from point F1 to point F3 is calculated. The vertical position of the heels is determined 1055. The vertical change in position of the heel is then calculated 1065 by the difference between initial point F1 and final point F3 and the difference F2 between the two points (F3−F1=F2). In various embodiments the horizontal axis linear distance reached by the patient is delivered or presented 1075 to the patient through a console or client device. Human pose estimation and additional algorithms may be used to interpret the images and image stream received via the camera or other sensors.

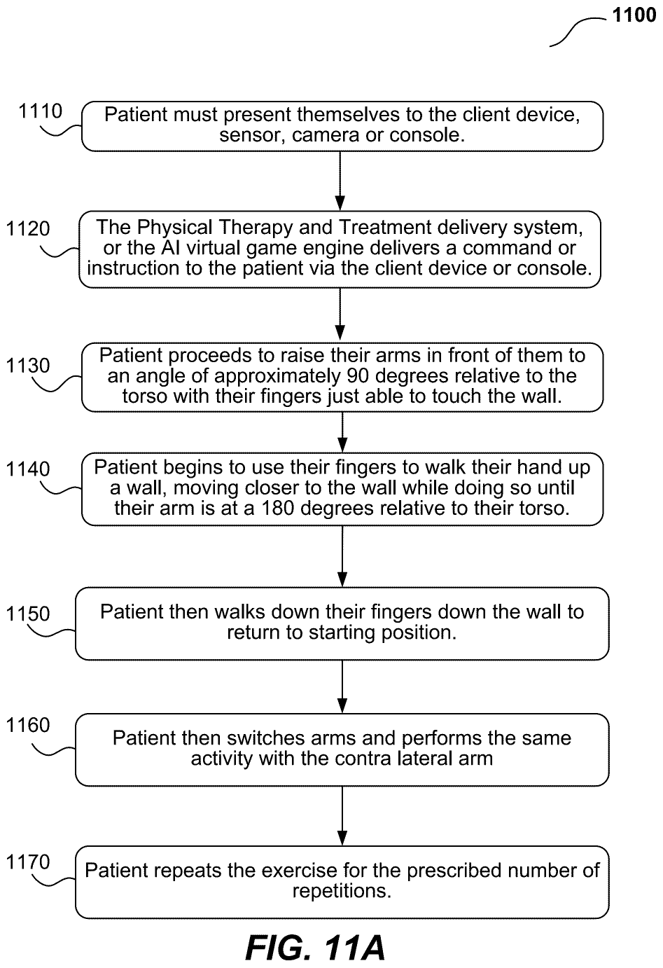

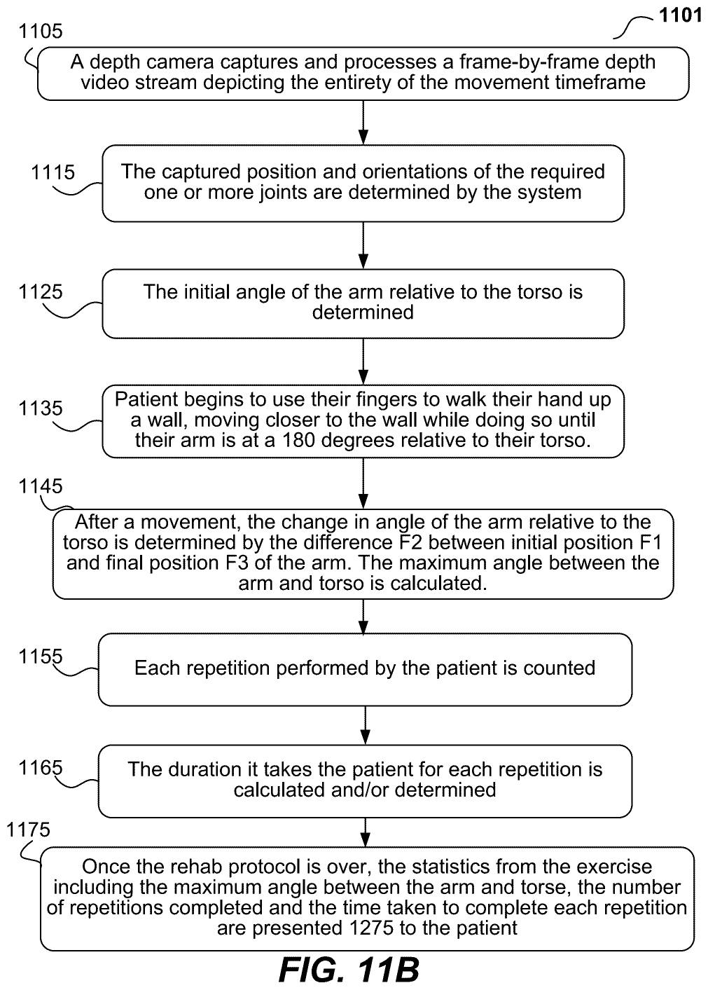

FIG. 11A presents one embodiment of an exemplary rehabilitation protocol, wall walking. The patient must present themselves 1110 in front of the console or client device and/or the camera, for example by standing in front of the camera, making sure that one or more of the following joints are visible to the depth camera, right heel, left heel, right hip, left hip, right wrist, left wrist, torso, neck, and head. Of course, the joints that must be visible to the camera depend on the requirements of each test. In this test the patient stands near a wall within their environment visible to the camera. Each test may require one, some, or all, of the joints to be visible. The PTTD system delivers a command or instruction 1120 to the user, which may be a voice instruction, the patient then raises their arms 1130 in front of them to an angle of approximately 90 degrees relative to the torso with their fingers just able to touch the wall. Patient then begins to use their fingers to walk their hand up the wall 1140, moving closer to the wall until their arm is at 180 degrees (or as high as possible) relative to the torso. Patient uses their fingers to then walk back down the wall and return to starting position 1150. Patient then switches arms and performs the same activity with the contra lateral arm 1160. Repeat for the prescribed number of repetitions for each arm 1170.

FIG. 11B presents one possible embodiment of the calculations carried out 1101 by the PTTD system and/or the AI engine for the functional reach test wall walking rehabilitation protocol discussed in FIG. 11A used for musculoskeletal (MSK) care. The camera captures and processes 1105 a frame-by-frame video stream depicting the entirety of the movement timeframe. One way this could be carried out is by the process as described in FIG. 8. The captured position and orientations of the required one or more joints are determined 1115 by the system. These joints may include any one or more of, and not limited to: the right heel, left heel, right hip, left hip, right wrist, left wrist, torso, neck and head. The initial angle of the arm relative to the torso is determined 1125. After a movement, the change in angle of the arm relative to the torso is determined 1135, by the difference F2 between initial position F1 and final position F3. The maximum angle between the arm and torso is calculated 1145. Each repetition performed by the patient is counted 1155. The duration it takes a patient for each repetition is also determined 1165. Once the rehab protocol is over, the statistics from the exercise including the maximum angle between the arm and torse, the number of repetitions completed, and the time taken to complete each repetition are presented 1175 to the patient.

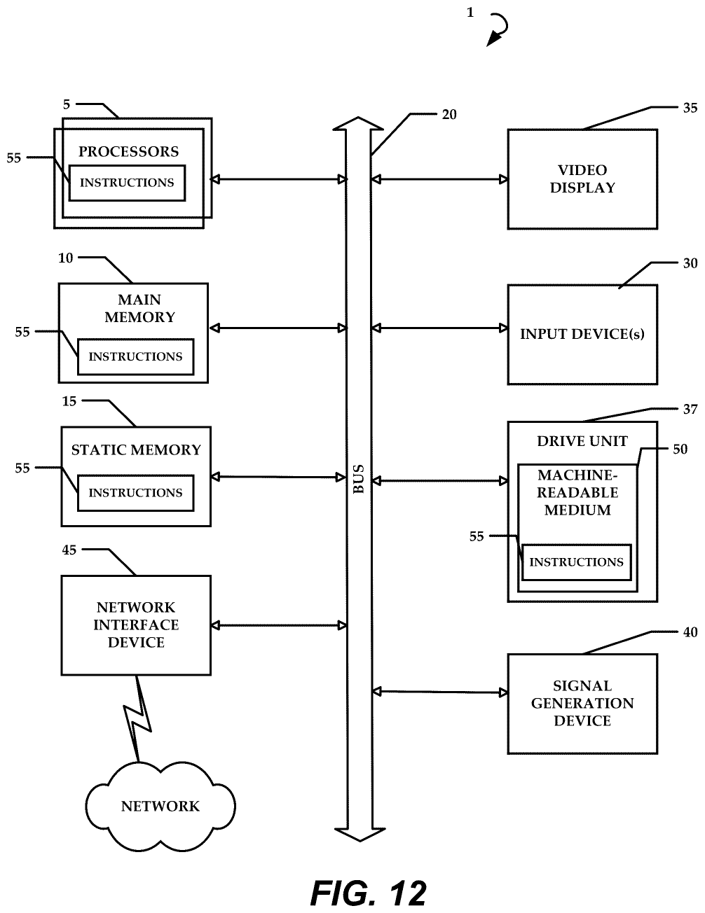

FIG. 12 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as an Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The disk drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network (e.g., network 115, see FIG. 1 or network 215, see FIG. 2) via the network interface device 45 utilizing any one of several well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that can store, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

One skilled in the art will recognize that Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized to implement any of the embodiments of the disclosure as described herein.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

FIG. 13 shows an exemplary method 1300 for remote physical therapy and guidance in accordance with embodiments of the present technology. The method begins with step 1305, where video is captured of an individual undertaking a predefined, directed activity. As described throughout this specification, the capturing is performed via one or more sensors on a device, which may include cameras. The individual undertakes a predefined, directed activity, which may be selected from a library containing a catalogue of physical therapy movements, routines, and regimens as described herein. The sensors on the device capture video of the individual's movements during this activity.

At step 1310, the captured video is processed on the device. This processing may be accomplished by human pose estimation, joint tracking, and motion analysis software that is integrated with a camera. The processing may occur locally on the client device or via the cloud.

Step 1315 involves analyzing the video using human pose estimation algorithms. The algorithm detects and tracks joint coordinates to determine body position, orientation, movement, angles, and velocity. This analysis utilizes artificial intelligence to determine body positioning and movement of the individual and estimates position of the individual in a space from a known point while estimating orientation of the individual's whole body, resulting in human body pose estimation.

At step 1320, time-stamped data is generated for the predefined, directed activity. The system processes the captured video and biomechanical data to create this time-stamped data. Changes in the spatial location are calculated into a multitude of variables such as, but not limited to, the number of repetitions, cadence, gait velocity, stride length, range of motion, and time to completion.

Step 1325 involves determining biomechanical parameters used to assess performance of the predefined, directed activity. These parameters may include metrics or other measures, the individual's health status, or other indicators of the individual's physical or mental state or wellbeing or an outcome pertaining falls risk, or functional health status.

At step 1330, feedback is provided via a user interface to the individual on their performance of the predefined, directed activity. The feedback is generated and displayed through a graphical user interface to show the performance assessment to the individual.

This method enables remote physical therapy by providing real-time analysis and feedback on an individual's movements and performance during prescribed physical therapy activities, allowing for effective rehabilitation and treatment.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

While various embodiments have been described above, they are presented as examples only, and not as a limitation. The descriptions are not intended to limit the scope of the present technology to the forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

1. A method for remote physical therapy and guidance, the method comprising:
capturing, via one or more sensors on a device, video of an individual undertaking a predefined, directed activity;
processing the captured video of the individual undertaking the predefined, directed activity on the device; and
the processing of the captured video comprising:
analyzing the captured video via a human pose estimation algorithm to detect and track joint coordinates to determine body position, orientation, movement, angles or velocity;
processing the captured video and biomechanical data to generate time-stamped data for the predefined, directed activity;
processing the time-stamped data to determine a biomechanical parameter used to assess performance of the predefined, directed activity; and
providing feedback via a user interface to the individual on the performance of the predefined, directed activity.

2. The method of claim 1, wherein the device comprises a mobile phone with a standard RGB camera, and wherein the human pose estimation algorithm comprises a commercially available pose estimation algorithm, a proprietary pose estimation algorithm, or a combination of commercially available and proprietary pose estimation algorithms.

3. The method of claim 2, further comprising executing the commercially available human pose estimation algorithm on the device for detecting and tracking joint coordinates of the individual in multiple dimensions including X, Y and Z axes during the performance of the predefined directed activity, generating time-stamped data describing segment and joint coordinates for the performance of the predefined activity, and processing the time-stamped data describing segment and joint coordinates through execution of the proprietary human pose estimation algorithm on the local computing device to determine the biomechanical parameter.

4. The method of claim 3, wherein the biomechanical parameter includes joint estimates and segment trajectories, joint and segment position, orientation, velocity, and motion kinematics including angle of knee flexion and velocity of elbow extension.

5. The method of claim 4, further comprising processing the time-stamped data and the biomechanical parameter on the device with the proprietary human pose estimation algorithm to determine one or more health, wellness, or clinical parameters including stance stability during balance, gait velocity during walking, or total time to complete the predefined, directed activity.

6. The method of claim 1, further comprising analyzing data from the human pose estimation algorithm on the device or transmitting the data to a cloud-based, on-premise, or hybrid compute, storage, and platform where additional artificial intelligence, analysis, and data interpretation may be conducted.

7. The method of claim 1, wherein analyzing the captured video via the human pose estimation algorithm includes age-and-gender based norm analysis, individual performance trends of the individual undertaking the predefined, directed activity across multiple sessions, individual historical analysis including consideration of other health considerations including prior physical therapy, medication, pain treatment, falls history, and scores on assessments, and comparative scores and movement features against normative datasets or prior personal baselines.

8. The method of claim 1, wherein the predefined, directed activity comprises therapeutic exercises selected from a stored database of clinically validated physical therapy routines categorized by anatomical region and injury type.

9. The method of claim 1, further comprising automatically comparing the individual's current biomechanical parameter values against personalized baseline data stored in a user profile to calculate performance improvement metrics.

10. The method of claim 1, further comprising executing anomaly detection algorithms to identify movement patterns deviating from expected therapeutic ranges and automatically generating electronic alerts for healthcare providers.

11. The method of claim 1, wherein the feedback comprises real-time visual overlays on the video display indicating joint position accuracy or computer-generated auditory instructions for movement correction during performance of the activity.

12. A system for remote physical therapy and assessment, the system comprising:
a mobile computing device having a processor, memory, and an RGB camera;
a graphical user interface configured to display personalized physical therapy instructions for therapeutic exercises targeting specific anatomical regions;
a motion analysis module stored in the memory and executable by the processor, the motion analysis module configured to capture video data of a user performing physical therapy exercises using the camera, apply human pose estimation algorithms to track joint positions and movements, calculate biomechanical metrics including joint angles and movement velocities from the tracked movements, and generate performance assessments based on comparison of the biomechanical metrics to therapeutic target ranges;
wherein the system provides immediate feedback to the user through the graphical user interface during exercise performance.

13. The system of claim 12, wherein the mobile computing device is a smartphone or tablet configured to execute the motion analysis module as software without requiring external motion capture hardware.

14. The system of claim 12, further comprising a cloud-based server system configured to store user profiles and aggregate performance data across multiple users, wherein data is encrypted at rest and in transit.

15. The system of claim 12, wherein the motion analysis module is configured to process video frames locally using computer vision algorithms without requiring network connectivity for joint tracking calculations.

16. The system of claim 12, further comprising an artificial intelligence engine executing machine learning algorithms configured to personalize exercise routines based on analysis of user performance data and individual baseline comparisons.

17. The system of claim 12, wherein the graphical user interface displays an animated avatar demonstrating proper exercise form with visual indicators for target joint positions and movement ranges.

18. The system of claim 12, further comprising integration capabilities with electronic medical record systems for automated transmission of patient performance data to healthcare providers.

19. A non-transitory computer-readable medium storing instructions that, when executed by a processor of a compute device, cause the compute device to:
initiate a physical therapy session by displaying exercise instructions for therapeutic routines on a screen of the compute device;
capture video of a user performing physical therapy exercises using a camera of the compute device;
process the captured video using human pose estimation algorithms executing on the compute device to extract joint coordinate data;
analyze the joint coordinate data using biomechanical calculation algorithms to determine compliance with prescribed exercise parameters including target joint angles and movement velocities;
generate performance metrics based on the analysis including completion accuracy and movement quality scores; and
provide immediate feedback to the user regarding exercise performance through visual displays on the screen or audio instructions via speakers of the compute device.

20. The non-transitory computer-readable medium of claim 19, wherein the instructions further cause the compute device to encrypt the performance metrics and transmit the encrypted data to a healthcare provider system for clinical review and electronic medical record integration.
